(19) United States
(12) Patent Application Publication
(10) Pub. No.: US 2026/0066090 A1
(43) Pub. Date: Mar. 5, 2026

(54) INFORMATION PROCESSING SYSTEM AND METHODS FOR CLINICAL VIDEO RETRIEVAL

(71) Applicant: The Curators of the University of Missouri, Columbia, MO (US)

(72) Inventors: Praveen Rao, Columbia, MO (US); Eduardo J. Simoes, Columbia, MO (US); Mihail Popescu, Columbia, MO (US); Mirna Becevic, Columbia, MO (US); Zhandi Liu, Columbia, MO (US)

(21) Appl. No.: 19/312,748

(22) Filed: Aug. 28, 2025

Related U.S. Application Data
(60) Provisional application No. 63/688,067, filed on Aug. 28, 2024.

(51) Int. Cl. G16H 30/20; G06F 16/71; G06F 16/738; G06F 16/78; G06F 16/783; G10L 15/26; H04N 21/845

(52) U.S. Cl. CPC G16H 30/20; G06F 16/71; G06F 16/738; G06F 16/783; G06F 16/7867; G10L 15/26; H04N 21/8456

(57) ABSTRACT

The present disclosure generally relates to an integrated approach for retrieving biomedical information from clinical video presentations. In particular, the present disclosure is directed to video retrieval systems and methods of text-video retrieval from clinical video presentations.

### CROSS REFERENCE TO RELATED APPLICATIONS

This application claims priority to and benefit of U.S. Provisional Patent Application No. 63/688,067, filed on Aug. 28, 2024, the disclosure of which is incorporated herein by reference in its entirety.

### STATEMENT OF GOVERNMENT SUPPORT

This invention was made with government support under DK092950 awarded by the National Institutes of Health. The government has certain rights in the invention.

### BACKGROUND OF THE DISCLOSURE

The present disclosure relates generally to an integrated approach for improving biomedical information retrieval from video presentations. In particular, the present disclosure is directed to video retrieval systems and methods of text-video retrieval of clinical video presentations.

With the increase in telehealth services and case-based learning sessions, medical professionals are gaining new knowledge and expertise via virtual consultations with experts. In this regard, clinical consultations are regularly recorded in the form of videos for medical education and improving patient outcomes. These videos contain a mixture of unstructured data including audio, video, and presentation materials. Telementoring and case-based learning sessions provide a rich repository of educational content accumulating thousands of hours of video recordings. Efficient video retrieval on these recordings is crucial for education and training of medical professionals as well as improving patient outcomes. Retrieving useful information from conversational clinical video recordings is challenging due to a mixture of unstructured data including audio, video, and didactic/case-based presentation materials. Furthermore, the conversations include clinical terminology that must be considered during retrieval.

The absence of effective search tools makes it challenging for healthcare providers to efficiently locate and access the specific information contained within a vast video repository. As a result, a significant portion of this valuable educational content remains underutilized and inaccessible to participants of telehealth and telementoring.

Accordingly, there exists a need for indexing clinical video recordings and search tools for retrieving information from clinical video recordings.

### BRIEF DESCRIPTION OF THE DISCLOSURE

The present disclosure generally relates to systems and methods for retrieving information from clinical video recordings. The present disclosure enables effective text-video retrieval of clinical video recordings containing case-based presentations and didactics. In particular, the systems and methods of the present disclosure provide a flexible framework for effective retrieval of clinical video recordings for text queries provided by users. During retrieval, given a user's text query, the system's framework outputs the top-k most relevant short clips in the video repository.

In one aspect, the present disclosure is directed to a computer-implemented clinical video retrieval system for searching and retrieving clinical videos and clinical text comprising: a data repository comprising clinical video recordings: an automatic speech recognition module configured to generate timestamped text transcriptions from the audio content of the clinical video recordings: an indexing module configured to (i) receive the timestamped text transcriptions, (ii) generate a record comprising a unique identifier, filename, timestamp, transcribed text, and a dense embedding vector of the transcribed text, and (iii) store the record in an index comprising an inverted index and a vector index: a query processing module configured to receive a text query from a user and generate a dense embedding vector of the text query using a transformer-based embedding model, wherein the query processing module optionally comprises a large language model configured to generate a set of dynamically rephrased query variants of the text query: a retrieval module configured to perform a keyword-based search using a ranking function on the inverted index and perform a semantic search using k-nearest neighbor (kNN) retrieval on the vector index, wherein results from the keyword-based search and the semantic search are combined using a weighted scoring function, and wherein candidate transcript segments are retrieved for each query variant: a cross-encoder reranking module configured to apply a cross-encoder reranker to the candidate transcript segments, user query and query variants to compute contextualized similarity scores and sort the candidate transcript segments based on the contextualized similarity scores to produce a ranked list of results: a video clip generating module configured to extract, for each ranked transcript segment, a video clip from the corresponding clinical video recording, the video clip beginning at the timestamp associated with the transcript segment and having a predefined duration; and a display module configured to present the ranked list of video clips and associated clinical text segments.

In one aspect, the present disclosure is directed to a computer-aided method for searching and retrieving clinical video clips and clinical text, the method comprising: (a) receiving a user query comprising a text input: (b) generating a dense embedding of the user query using at least one transformer-based embedding model: (c) retrieving, from a repository of clinical video recordings, a plurality of transcript segments, each transcript segment comprising a timestamped transcription of a portion of a clinical video, wherein the retrieval comprises: (i) performing a keyword-based search using a lexical ranking function to identify transcript segments relevant to the user query: (ii) performing a semantic search by computing similarity between the dense embedding of the user query and precomputed dense embeddings of the transcript segments to identify semantically relevant transcript segments: (iii) combining results from the keyword-based search and the semantic search using a weighted scoring function to generate a set of candidate transcript segments: (d) reranking the set of candidate transcript segments using a cross-encoder model to compute contextualized similarity scores between the user query and each candidate transcript segment, and sorting the candidate transcript segments based on the contextualized similarity scores: (e) extracting, for each reranked transcript segment, a corresponding clinical video clip from the clinical video recordings, wherein the video clip is generated based on the timestamp associated with the transcript segment: (f) outputting to a user interface the corresponding clinical video clips as video results, the reranked transcript segments as clinical text results, and combinations thereof.

In one aspect, the present disclosure is directed to a computer-aided process for indexing clinical video recordings, the process comprising: receiving a plurality of clinical video recordings, the clinical video recordings comprising audio, video, and presentation materials: transcribing audio of each clinical video recording of the plurality of clinical video recordings using automatic speech recognition (ASR) software to generate timestamped text transcripts corresponding to segments of the video recordings: generating dense vector embeddings for each segment of the transcribed text using a transformer-based deep learning model, wherein each dense vector embedding represents semantic content of the corresponding transcript segment: constructing an index in a search engine system, the index comprising an inverted index for keyword-based search, a vector index, and metadata for each transcript segment including a unique identifier, a filename, a timestamp, transcribed text, and the corresponding dense embedding vector: storing the index; and enabling retrieval of relevant video clips.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is an illustration depicting an exemplary embodiment of the video retrieval system of the present disclosure.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure enables effective text-video retrieval of clinical video recordings containing case-based presentations and didactics. In particular, the systems and methods of the present disclosure provide a flexible framework for effective retrieval of clinical video recordings for text queries provided by users. During retrieval, given a user's text query, the framework outputs the top-k most relevant short clips in the video repository.

In one aspect, the present disclosure is directed to a computer-implemented clinical video retrieval system for searching and retrieving clinical videos and clinical text comprising: a clinical data repository comprising clinical video recordings: an automatic speech recognition module configured to generate timestamped text transcriptions from the audio content of the clinical video recordings: an indexing module configured to (i) receive the timestamped text transcriptions, (ii) generate a record comprising a unique identifier, filename, timestamp, transcribed text, and a dense embedding vector of the transcribed text, and (iii) store the record in an index comprising an inverted index and a vector index: a text query processing module configured to receive a text query from a user and generate a dense embedding vector of the text query using a transformer-based embedding model, wherein the text query processing module optionally comprises a large language model configured to generate a set of dynamically rephrased text query variants of the text query from the user: a retrieval module configured to perform a keyword-based search using a ranking function on the inverted index and perform a semantic search using k-nearest neighbor (kNN) retrieval on the vector index, wherein results from the keyword-based search and the semantic search are combined using a weighted scoring function, and wherein candidate transcript segments are retrieved for each text query variant: a cross-encoder reranking module configured to apply a cross-encoder reranker to the candidate transcript segments, text query from the user and text query variants to compute contextualized similarity scores and sort the candidate transcript segments based on the contextualized similarity scores to produce a ranked list of results: a video clip generating module configured to extract, for each ranked transcript segment, a video clip from the corresponding clinical video recording, the video clip beginning at the timestamp associated with the transcript segment and having a predefined duration; and a display module configured to present the ranked list of video clips and associated clinical text segments.

The clinical data repository includes video recordings containing audio discussions between different participants involving clinical terminology, slide presentations with textual information, and images. The clinical video repository can also include telehealth or telementoring session recordings. Each clinical video recording of the clinical video repository ranges from about 0.4 GB to about 0.8 GB. The term, “video clip” is used herein according to its ordinary meaning to refer to a short segment of a longer video recording.

Any suitable automatic speech recognition (ASR) model is used to generate timestamped text transcriptions from the audio content of the clinical video recordings. Suitable ASR models include MICROSOFT OneDrive, GOOGLE Cloud Speech-to-Text, AMAZON Transcribe, IBM Watson Speech to Text, and Deepgram.

Any suitable indexing module is used to receive the timestamped text transcriptions, generate a record comprising a unique identifier, filename, timestamp, transcribed text, and a dense embedding vector of the transcribed text, and store the record in an index comprising an inverted index and a vector index. Suitable indexing models include APACHE SOLR, Elasticsearch, and MongoDB. APACHE SOLR is an open-source software with full-text indexing and search capabilities. Elasticsearch is another open source, distributed search and analytics engine that stores structured, unstructured, and vector data. MongoDB provides vector representations of data to perform semantic search, build recommendation engines, and design question and answer systems. For indexing transcribed text, the input is provided in the form of a JSON document where each record includes at least a unique identifier, a filename, a timestamp, transcribed text, and an embedding vector of the text. When APACHE SOLR is used as the indexing module the index constructed by it includes an inverted index and a Hierarchical Navigable Small World (HNSW) vector index. The indexing schema incorporates Best Match 25 (BM25)-based keyword search fields along with dense vector representations to support semantic search. Raw transcriptions with timestamps enable context-aware retrieval and precomputed dense vector embeddings for each segment enable k-nearest neighbor (kNN) retrieval for similarity search. The timestamps of the retrieved text is used to locate the start time of relevant video clips shown as output to the user during clinical video retrieval. Elasticsearch and MongoDB are used as similarly described for APACHE SOLR.

The clinical video retrieval system supports different search paradigms, ranging from keyword-based retrieval to hybrid neural search with reranking. The first stage of retrieval uses a ranking function. Given a user query, the indexing module retrieves the most relevant transcript segments based on the ranking similarity score. To improve retrieval effectiveness, the system further integrates keyword retrieval with neural search.

In one embodiment, the indexing module is further configured to generate dense embedding vectors using an ensemble of multiple transformer-based embedding models, and to store the multiple embeddings for each transcript segment in the index. Suitable transformer-based embedding models include S-PubMedBERT, fine-tuned S-PubMedBERT using the MedQA-USMLE dataset, BGE, Bio ClinicalBERT, E5, DistilClinicalBERT, TinyClinicalBERT, MedBERT, BlueBERT, and Clinical ModernBERT.

Any suitable cross-encoder reranking module is used to apply a cross-encoder reranker to the candidate transcript segments, user text query, and text query variants to compute contextualized similarity scores and sort the candidate transcript segments based on the contextualized similarity scores to produce a ranked list of results. Suitable cross-encoder rerankers include MS-MARCO-MiniLM, BGE reranker, nli-deberta-v3-large, stsb-distilroberta-base, jina-reranker-v1-turbo-en, ColBERT, and Qwen2-7B.

The predefined duration of the video clip begins at the timestamp associated with the transcript segment and has a predefined duration. The predefined duration of the video clip begins at the timestamp associated with the transcript segment and has a predefined duration. The predefined duration of each video clip depends on the user query and resulting video clip retrieval. Generally, the video clip has a duration less than the full video recording from which the video clip is derived. Thus, the video clip can be seconds in duration, minutes in duration, and even hours in duration.

The display module is further configured to present metadata. Metadata that is presented includes a filename, a timestamp, and combinations thereof.

In one aspect, the present disclosure is directed to a computer-aided method for searching and retrieving clinical video clips and clinical text, the method comprising: (a) receiving a user query comprising a text input: (b) generating a dense embedding of the user query using at least one transformer-based embedding model: (c) retrieving, from a repository of clinical video recordings, a plurality of transcript segments, each transcript segment comprising a timestamped transcription of a portion of a clinical video, wherein the retrieval comprises: (i) performing a keyword-based search using a lexical ranking function to identify transcript segments relevant to the user query: (ii) performing a semantic search by computing similarity between the dense embedding of the user query and precomputed dense embeddings of the transcript segments to identify semantically relevant transcript segments: (iii) combining results from the keyword-based search and the semantic search using a weighted scoring function to generate a set of candidate transcript segments: (d) reranking the set of candidate transcript segments using a cross-encoder model to compute contextualized similarity scores between the user query and each candidate transcript segment, and sorting the candidate transcript segments based on the contextualized similarity scores: (e) extracting, for each reranked transcript segment, a corresponding clinical video clip from the clinical video recordings, wherein the video clip is generated based on the timestamp associated with the transcript segment: (f) outputting to a user interface the corresponding clinical video clips as video results, the reranked transcript segments as clinical text results, and combinations thereof.

In some embodiments, the computer-aided method further includes enhancing retrieval accuracy by dynamically generating one or more rephrased variants of the user query using a large language model, and repeating steps (b) through (f) for each rephrased variant, wherein results from multiple query variants are combined using a round-robin or interleaving strategy with duplicate removal.

The transcript segments in the computer-aided method are indexed in a search engine comprising an inverted index for keyword-based search and a vector index for semantic search. Suitable indexing models include APACHE SOLR, Elasticsearch, and MongoDB.

The dense embeddings of the transcript segments in the computer-aided method are generated using an ensemble of multiple transformer-based embedding models. Suitable transformer-based embedding models include S-PubMedBERT, fine-tuned S-PubMedBERT using the MedQA-USMLE dataset, BGE, Bio ClinicalBERT, E5, DistilClinicalBERT, TinyClinicalBERT, MedBERT, BlueBERT, and Clinical ModernBERT.

Suitable cross-encoder models used in the computer-aided method include MS-MARCO-MiniLM, BGE reranker, nli-deberta-v3-large, stsb-distilroberta-base, jina-reranker-v1-turbo-en, ColBERT, and Qwen2-7B.

The transcript segments are generated using automatic speech recognition (ASR) software. Suitable ASR models include MICROSOFT OneDrive, GOOGLE Cloud Speech-to-Text, AMAZON Transcribe, IBM Watson Speech to Text, and Deepgram. The clinical data repository includes video recordings containing audio discussions between different participants involving clinical terminology, slide presentations with textual information, and images. The clinical video repository can also include telehealth or telementoring session recordings.

The computer-aided method provides output of video clips of a predefined duration. The predefined duration of the video clip begins at the timestamp associated with the transcript segment and has a predefined duration. The predefined duration of each video clip depends on the user query and resulting video clip retrieval. Generally, the video clip has a duration less than the full video recording from which the video clip is derived. Thus, the video clip can be seconds in duration, minutes in duration, and even hours in duration.

In one aspect, the present disclosure is directed to a computer-aided process for indexing clinical video recordings, the process comprising: receiving a plurality of clinical video recordings, the clinical video recordings comprising audio, video, and presentation materials: transcribing audio of each clinical video recording of the plurality of clinical video recordings using automatic speech recognition (ASR) software to generate timestamped text transcripts corresponding to segments of the video recordings: generating dense vector embeddings for each segment of the transcribed text using a transformer-based deep learning model, wherein each dense vector embedding represents semantic content of the corresponding transcript segment: constructing an index in a search engine system, the index comprising an inverted index for keyword-based search, a vector index, and metadata for each transcript segment including a unique identifier, a filename, a timestamp, transcribed text, and the corresponding dense embedding vector: storing the index; and enabling retrieval of relevant video clips.

Suitable ASR models include MICROSOFT OneDrive, GOOGLE Cloud Speech-to-Text, AMAZON Transcribe, IBM Watson Speech to Text, and Deepgram.

The transcript segments in the computer-aided process are indexed in a search engine system comprising an inverted index for keyword-based search and a vector index for semantic search. Suitable indexing models include APACHE SOLR, Elasticsearch, and MongoDB.

Suitable transformer-based deep learning models of the computer-aided process for indexing clinical video recordings include S-PubMedBERT, fine-tuned S-PubMedBERT using the MedQA-USMLE dataset, BGE, Bio ClinicalBERT, E5, DistilClinicalBERT, Tiny ClinicalBERT, MedBERT, BlueBERT, Clinical ModernBERT, and combinations thereof.

The enabling retrieval of relevant video clips of the computer-aided process for indexing clinical video recordings includes receiving a user text query, generating a dense embedding of the user text query using a transformer-based model, retrieving candidate transcript segments using a combination of keyword-based search and neural vector-based search, reranking the candidate results using a cross-encoder model to compute contextualized similarity scores between the query and each candidate transcript segment, reranking the set of candidate transcript segments using a cross-encoder model to compute contextualized similarity scores between the user query and each candidate transcript segment, and sorting the candidate transcript segments based on the contextualized similarity scores, and outputting a ranked list of video clips, wherein each video clip corresponds to a segment of a clinical video recording starting at the timestamp associated with a top-ranked transcript segment.

The computer-aided process for indexing clinical video recordings can further include dynamically rephrasing the user text query using a large language model to generate a plurality of semantically similar text query variants, performing retrieval and reranking for each semantically similar text query variant.

### Examples

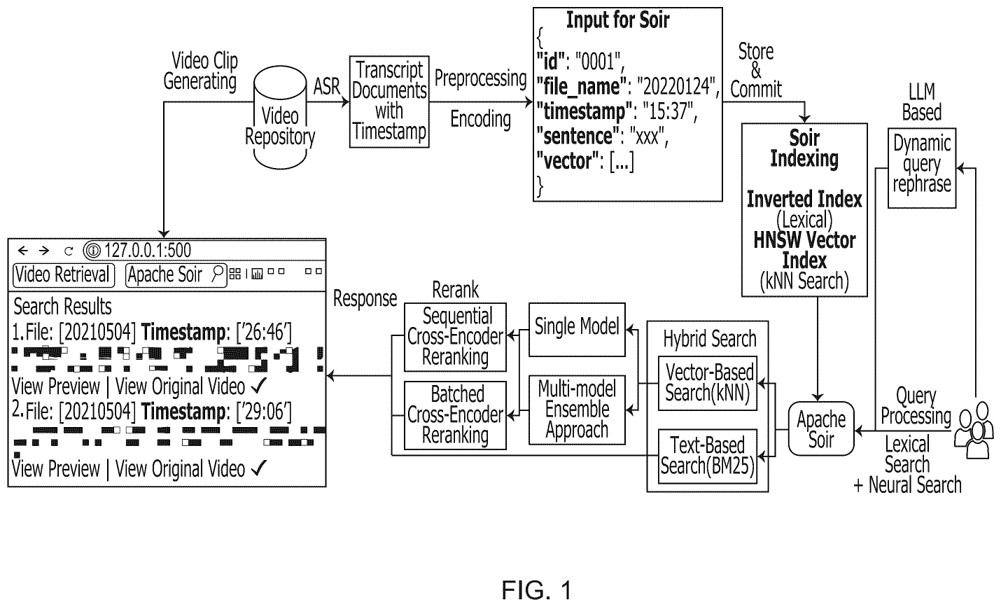

This Example describes the design of the video retrieval system and the steps of indexing, candidate retrieval, and reranking using different deep learning approaches. FIG. 1 illustrates an overview of the system and components.

The video repository used in this Example contained 66 videos for case-based presentations regarding diabetes patients and didactics from the Show-Me Extension for Community Healthcare Outcomes (ECHO) sessions. (The study was approved under MU Institutional Review Board (IRB) No. 2098690.) The maximum and minimum size of the videos was 0.8 GB and 0.4 GB, respectively. The Show-Me Extension for Community Healthcare Outcomes (ECHO) is a state-funded program at the University of Missouri (MU) to advance telehealth for communities that do not have access to expert physicians. During an ECHO session, experts and primary care physicians (PCP) collaborate through case-based learning over Zoom. These video sessions are recorded and shared with other professionals for knowledge dissemination and education. The video recordings contain expert knowledge and feedback on complex cases dealt with by PCPs.

The video recordings contained discussions between different participants involving the analysis of patient cases as well as slide presentations containing expert knowledge such as new treatments. The recordings were transcribed using Microsoft OneDrive's built-in ASR method, which generated timestamped transcripts in text. For example, the following was an output text produced via ASR indicating the time in minutes and seconds along with the transcription of the audio:
[10:23 It's important to remember that type one patients still need to have their basal insulin.]

Thus, instead of indexing the raw videos, transcripts of the videos were indexed with the aim to support text queries such as “What is the best treatment for diabetic neuropathy?”

The textual data was indexed using Apache Solr, an open-source software with full-text indexing and search capabilities. For Apache Solr to index the transcribed text, the input was provided in the form of a JSON document where each record contained: ID, filename, timestamp, transcribed text, embedding vector of the text. Based on the length of the recording, a video recording produced 100's of such records. The indexes constructed by Apache Solr included an inverted index and a Hierarchical Navigable Small World (HNSW) vector index. The indexing schema incorporated Best Match 25 (BM25)-based keyword search fields along with dense vector representations to support semantic search. Raw transcriptions with timestamps enabled context-aware retrieval and precomputed dense vector embeddings for each segment enabled k-nearest neighbor (kNN) retrieval for similarity search. The timestamps of the retrieved text were used to locate the start time of relevant video clips to be shown as output to the user during video retrieval. Five different ways of generating the text embeddings for indexing were investigated, which are discussed below.

The video retrieval system was designed to support different search paradigms, ranging from keyword-based retrieval to hybrid neural search with reranking. The first stage of retrieval used Apache Solr's BM25 ranking function. Given a user query, Apache Solr retrieves the most relevant transcript segments based on the BM25 similarity score. To improve retrieval effectiveness of the system BM25 keyword retrieval was integrated with neural search. The neural component involves indexing dense embeddings generated from transformer-based models such as S-PubMedBERT, fine-tuned S-PubMedBERT using the MedQA-USMLE dataset, BGE, Bio ClinicalBERT, and E5. (See Table II for more details.)

When a user submits a text query, its embedding representation is computed in real-time and used to retrieve the k-nearest neighbor documents via Apache Solr's approximate nearest neighbor (ANN) retrieval. The candidate results from BM25 and neural search were combined using a weighted scoring function. After that, reranking is performed to output the final results to the user. To refine the candidate results, a cross-encoder reranker is applied. The rerankers (cross-encoder) used were MS-MARCO-MiniLM and BGE reranker. The reranker scores each query-document pair by computing contextualized similarities, prioritizing transcriptions most relevant to the user query. Finally, a 3-minute video clips was extracted starting from the timestamps obtained during retrieval. The overall steps are shown in Algorithm 1.

Algorithm 1. General text-video retrieval

| Input: Set of videos V, input query q, value of K in top-K results | | |
|---|---|---|
| Output: A globally ranked list of K video clips | | |
| 1: | | Generate dense embedding of q (based on the model used for |
| indexing) | | |
| 2: | | Run keyword-based (BM25) and kNN semantic search for q to |
| retrieve initial text results with video file names and timestamps | | |
| | 3: | Apply cross-encoder reranking sequentially to the retrieved results |
| | for q // One-pass ranking with no iterative modifications | |
| 4: | | Sort results by cross-encoder score in descending order |
| 5: | | Let G denote the top-K sorted results |
| 6: | | Initialize an empty list C for video clips |
| 7: | | for each result r ϵ G do |
| 8: | | Extract file name and timestamp from r |
| | 9: | Generate a 3-minute video clip starting at the timestamp from the |
| | corresponding video file | |
| 10: | | Add clip to C |
| 11: | | return C |

To improve video retrieval performance, two approaches were used. The first approach was an ensemble method of generating dense embeddings to enhance precision and efficiency. During indexing, the text transcriptions were fed to multiple embedding models (e.g., S-PubMedBERT, BGE-Large, E5-Base) to generate dense embeddings. These were stored in Apache Solr during index construction. During query processing (see, Algorithm 2), the dense embeddings of a query q were also constructed via the same embedding models. The top-k results based on BM25 and neural search (or hybrid search) of Apache Solr were obtained for each dense embedding of the query. The results were first merged and duplicates were removed. After that, reranking was performed using a cross-encoder to obtain the top-k list of text transcriptions (or documents). The final results were computed similar to Lines 7-10 in Algorithm 1.

Algorithm 2. Ensemble-based dense embeddings for text-video retrieval.

Input: Query q, list of embedding models {M₁,..., Mₙ}, cross-encoder model B, K
Output: A ranked list of text transcriptions (or documents) with timestamps

| 1: | | Extract text from videos to form a database D |
|---|---|---|
| 2: | | for each model Mᵢ ϵ {M₁,. . . , Mₙ} do |
| 3: | | Encode query q using Mᵢ to obtain embedding eᵢ |
| | 4: | Let rᵢ denote the top-K documents retrieved by Solr corresponding |
| | to Mᵢ using eᵢ on D | |
| 5: | | Let G ← ∪ᵢ rᵢ // merging and deduplication |
| 6: | | for each document d ϵ G do |
| 7: | | Let s_{d} ← B(q, d) // compute re-ranking score using cross-encoder |
| 8: | | Sort G by s_{d} in descending order |
| 9: | | return G |

The second approach was based on DQR to improve recall. The overall steps are shown in Algorithm 3. Using an LLM, DQR generates a set of semantically similar query variants Q for the input query q by incorporating domain-specific medical terms where relevant. Each query variant in Q was processed as before using hybrid search with results reranked per query using a cross-encoder. To combine these multi-query results, a round-robin interleaving strategy collected the i-th ranked result from each query's list at each iteration, sorted the block by cross-encoder score, and appended it to a global list, removing duplicates. Video clips were generated from the ranked text transcriptions similar to Lines 7-10 in Algorithm 1. While the ensemble method (first approach) prioritized efficiency and precision using diverse embeddings. DQR (second approach) focused on recall by utilizing query diversity, at the expense of increased computational cost.

Algorithm 3. Text-video retrieval with DQR and round-robin reranking.

Input: Input query q, K, cross-encoder model B
Output: A globally ranked list of text transcriptions (or documents) with timestamps

| | |
|---|---|
| 1: | Using an LLM, generate a set of rephrased queries Q = {q} ∪ |
| | {LLM-rephrased variants of q} |
| 2: | for each query qᵢ ϵ Q do |
| 3: | Do Step 2 - 4 in Algorithm 1 |
| 4: | Store top-K results in a list R_{qi} |
| 5: | Initialize an empty global list G for storing ranked results |
| 6: | Set i = 0 (rank index for round-robin) |
| 7: | repeat |
| 8: | Form a block b by taking the i-th result from each R_{qi} (if available) |
| 9: | Sort b by cross-encoder score in descending order obtained by B |
| 10: | Append results from b to G, removing duplicates |
| 11: | Increment i = i + 1 |
| 12: | until Size of G reaches K or all results from all R_{qi} are exhausted |
| 13: | return G |

### Experimental Setup

To evaluate the effectiveness of the video retrieval system, experiments were conducted on a video repository and the mean average precision at k (mAP@k) metric was computed. The experiments were designed to assess different indexing and search methodologies based on their ability to retrieve relevant results for clinical queries of interest to physicians.

Physicians formulated eight representative questions (Table 1), each corresponding to a distinct topic discussed in one or more Show-Me Extension for Community Healthcare Outcomes (ECHO) meetings. The questions served as test queries for the system. The questions are listed in Table I.

TABLE 1
List of Test Queries

| Query ID | Query |
|---|---|
| 1 | What is the risk of cardiovascular disease among diabetic |
| | persons? |
| 2 | What is the best treatment of diabetic neuropathy? |
| 3 | Is an insulin pump effective in managing diabetes? |
| 4 | What are the routine medications to help manage diabetes? |
| 5 | How is Type 2 diabetes classified? |
| 6 | Do glucose sensors work in the control of diabetes? |
| 7 | What is the recommended diet for diabetic persons? |
| 8 | What are the most prescribed non-insulin medications? |

For each query, the retrieval step was performed using both normal keyword search and neural search in various configurations with different rerankers and DQR (using the DeepSeek API). The top 25 retrieved results for each query were collected and evaluated based on two criteria: (1) Manual Relevance Assessment Manual Relevance Assessment by Physicians and (2) Automatic Keyword-based Evaluation. In Manual Relevance Assessment, the physicians provided ground truth relevance judgments for a subset of the retrieved results. This partial manual evaluation ensured expert-driven validation of the retrieved results. In Automatic Keyword-Based Evaluation, the existence of key medical terms expected as part of the relevant responses was checked. Keywords that should appear in the answers to each question were predefined based on expert knowledge. Using the ground truth relevance scores from manual assessment and the keyword-matching results from automatic evaluation, mAP@5, mAP@10, mAP@15, mAP@20, and mAP@25 were computed for each retrieval method.

Multiple model configurations were systematically tested from traditional keyword search to hybrid neural search approaches with rerankings and DQR. Table II shows the different methods used for text-video retrieval.

TABLE II
List of evaluated methods for indexing, retrieval and reranking.

| | | |
|---|---|---|
| Method | Indexing and Retrieval | Reranking |

BM25-Based Keyword Search

| | | |
|---|---|---|
| C0 | BM25 standard keyword-based | — |
| | search | |

Fine-tuned Neural Search with Reranking

| | | |
|---|---|---|
| C1 | S-PubMedBERT^{a} fine-tuned on | MS-MARCO-MiniLM^{b} |
| | MedQA-USMLE | |
| C2 | S-PubMedBERTa | MS-MARCO-MiniLM^{b} |
| C3 | S-PubMedBERT^{a} | BGE-Reranker^{c} |
| C4 | BGE-Large^{d} | BGE-Reranker^{c} |
| C5 | E5-Base^{e} | MS-MARCO-MiniLM^{b} |
| C6 | BioClinicalBERT^{f} | MS-MARCO-MiniLM^{b} |

Ensemble Neural Search with Reranking

| | | |
|---|---|---|
| C7 | Combined embeddings from | MS-MARCO-MiniLM^{b} |
| | S-PubMedBERT^{a}, BGE-Large^{d}, | |
| | E5-Base^{e} | |

Dynamic Query Rephrasing with Hybrid Search and Reranking

| | | |
|---|---|---|
| C8 | E5-Base^{e} | MS-MARCO-MiniLM^{b} |
| C9 | S-PubMedBERT^{a} | MS-MARCO-MiniLM^{b} |
| C10 | BGE-Large^{d} | BGE-Reranker^{c} |

^{a}pritamdeka/S-PubMedBert-MS-MARCO,
^{b}cross-encoder/ms-marco-MiniLM-L-12-v2,
^{c}BAAI/bge-reranker-v2-m3,
^{d}BAAI/bge-large-en-v1.5,
^{e}intfloat/e5-base-v2,
^{f}emilyalsentzer/Bio_ClinicalBERT

mAP@k Results

Each method was evaluated using the eight test queries (Table I) and compared using the mAP score, which is widely used to evaluate information retrieval systems. The results are shown in Table III for mAP@5, mAP@10, mAP@15, mAP@20, and mAP@25. The total wall-clock time for each method was also reported including the LLM API call time for C8, C9, and C10. Method C7 that used combined embeddings for indexing and retrieval and cross-encoder for reranking achieved the best performance for k=5, k=10, and k=15. However, for k=20 and k=25, method C10 that used BGE for indexing and reranking along with LLM-based DQR achieved the best performance. Note that methods C8, C9, and C10 had higher wall-clock time due to the use of LLM API call, which may be lowered if LLM models were run locally on a powerful GPU server.

TABLE III
mAP@k Score (Best mAP score in bold).

| | | | | | | Response Time |
|---|---|---|---|---|---|---|
| Bi-encoder | | | | | | (LLM API |
| and Reranker | mAP@5 | mAP@10 | mAP@15 | mAP@20 | mAP@25 | call time) |
| C0 | 0.6795 | 0.6534 | — | — | — | 2 s |
| C1 | 0.8616 | 0.8388 | 0.8221 | 0.7682 | 0.7530 | 2 s |
| C2 | 0.9319 | 0.8915 | 0.8752 | 0.8619 | 0.8465 | 2 s |
| C3 | 0.9285 | 0.9054 | 0.8962 | 0.8842 | 0.8702 | 7 s |
| C4 | 0.9408 | 0.9163 | 0.9035 | 0.8883 | 0.8727 | 8 s |
| C5 | 0.9389 | 0.8991 | 0.8885 | 0.8779 | 0.8706 | 3 s |
| C6 | 0.8512 | 0.8250 | 0.7947 | 0.7723 | 0.7381 | 2 s |
| C7 | 0.9859 | 0.9432 | 0.9178 | 0.8988 | 0.8760 | 4 s |
| C8 | 0.9173 | 0.8608 | 0.8467 | 0.8416 | 0.8347 | 13 s (10 s) |
| C9 | 0.9510 | 0.9030 | 0.8836 | 0.8710 | 0.8579 | 12 s (9 s) |
| C10 | 0.9693 | 0.9396 | 0.9172 | 0.9046 | 0.8877 | 17 s (10 s) |

These experimental results highlight key insights into the effectiveness of different retrieval strategies for clinical video recordings. It is worth noting that sentence transformer outperformed traditional BERT based models, such as ClinicalBERT, in handling conversational text for question-answering tasks likely because sentence transformers (e.g., BAAI/bge-large-en-v1.5) are optimized for semantic similarity tasks and can generate dense vector representations that better represent contextual meanings across sentences. However, models like ClinicalBERT are primarily pre-trained on structured clinical notes and are not suitable for unstructured, dynamic nature of spoken dialogue in medical discussions. Conversational data often involve incomplete sentences, implicit references, and turn-taking, making them suitable for models trained for sentence embedding-based retrieval.

These findings also emphasize the poor performance of traditional BM25-based keyword search for question-answering retrieval. Keyword-based methods rely on exact term matching and cannot effectively handle wording variations, resulting in low recall when the user phrases the question differently than how it appears in the transcript. They may retrieve another question sentence that is similar to the question (input query). However, neural search methods, especially those that leverage sentence transformers, can better understand the semantic relationship between questions and answers retrieving results that contain relevant answers.

Among the retrieval configurations tested in this Example, the ensemble neural search showed the best performance up to mAP@15, outperforming individual neural search methods with dynamic query expansion. By leveraging multiple embedding models, ensemble retrieval benefits from complementary representations, enhancing recall and precision at lower cutoffs. However, dynamic query expansion using neural search surpasses other methods after mAP@15, suggesting that query expansion helps retrieve more relevant sentences as the result set increases. The ability to dynamically rephrase and expand user queries can improve the matching of responses, especially in cases where clinical terms have many related or more precise or ambiguous representations.

### Retrieval Time

For C10, the total time was measured on two different GPU hardware by increasing the query-document pairs. The number of documents was fixed at 60. The number of queries was varied from 3, to 21, to 50, and to 167. Two techniques were applied. The first technique computed the cross-encoder score for (query, document) pairs one-at-a-time on the GPU (referred to as “Sequential reranking”). The second technique computed the score on a set of (query, document) pairs for the same document as a batched input (referred to as “Batched reranking”). Table IV reports the total wall-clock time for processing queries with sequential reranking and batched reranking for different number of query-document pairs on Mac M2 Pro (16 GB) and Nvidia RTX A4000. (The reranking time is also reported.)

TABLE IV
Comparison of Sequential and Batched Reranking
Total Execution Time on Different Hardware

| | Query-Doc. | Sequential | Batched |
|---|---|---|---|
| Hardware | Pairs | (reranking time) | (reranking time) |
| M2 Pro | 180 | 25.81 s (8.37 s) | 29.40 s (7.44 s) |
| | 1,260 | 91.84 s (59.39 s) | 133.93 s (54.95 s) |
| | 3,000 | 309.93 s (190.15 s) | 308.51 s (133.15 s) |
| | 10,020 | 1,520.36 s (1133.25 s) | 1,073.58 s (503.64 s) |
| RTX | 180 | 16.96 s (3.48 s) | 14.46 s (0.86 s) |
| A4000 | 1,260 | 53.08 s (26.82 s) | 46.42 s (6.05 s) |
| | 3,000 | 116.38 s (67.59 s) | 74.77 s (14.63 s) |
| | 10,020 | 331.29 s (250.54 s) | 188.95 s (54.48 s) |

On the M2 Pro, sequential reranking outperformed batched reranking for smaller workloads, achieving 25.81 seconds vs. 29.40 seconds at 180 pairs and 91.84 seconds vs. 133.93 seconds at 1260 pairs, due to lower overhead and efficient per-query GPU utilization. Batched reranking surpassed sequential reranking only at 3,000 pairs (308.51 s vs. 309.93 s), with a significant 29.4% advantage at 10,000 pairs (1073.58 s vs. 1520.36 s), providing scalability for larger datasets. In contrast, on the RTX A4000, batched reranking excelled across all scales: 14.46 seconds vs. 16.96 seconds at 180 pairs (14.7% faster) up to 188.95 seconds vs. 331.29 seconds at 10,020 pairs (43.0% faster), highlighting its consistent efficiency.

The architectural differences of the two GPU hardware dictates the observed performance results. The M2 Pro's unified memory limits batch sizes, favoring sequential processing for n<3, 000, while the RTX A4000's dedicated VRAM and superior compute enhances batching from the outset. For the M2 Pro, sequential reranking suits smaller workloads, with batching preferred beyond 3,000 pairs. On RTX A4000, batching is the clear choice regardless of size, offering substantial speedup and scalability for transformer-based reranking tasks.

The methodology described in this Example focused on indexing conversational transcripts, retrieving candidate clips, and reranking results using state-of-the-art sentence-transformer models. Additionally, retrieval performance was improved through LLM-powered DQR.

Experimental results show that the best method for mAP@5, mAP@10, and mAP@15 leveraged the BGE in conjunction with the MS MARCO cross-encoder reranker. For larger retrieval sets (mAP@20 and mAP@25), the best approach used BGE for both indexing, candidate retrieval, and reranking along with LLM-based DQR. With its 326M parameters, BGE effectively captures complex semantic structures for both short queries and lengthy conversations, significantly improving information retrieval performance. Additionally, LLM-based DQR enhanced retrieval by generating diverse, semantically similar queries that better align with user intent. Rephrased queries also enabled techniques like query expansion, which refines retrieval models, boosting system robustness and personalization. Finally, analysis of the total execution time of different retrieval approaches (i.e., sequential reranking and batched reranking) and introduced additional performance improvements for reranking.

The systems and methods of the present disclosure provide a flexible framework for effective retrieval of clinical video recordings for text queries provided by users. The methods provided herein focus on indexing conversational transcripts, retrieving candidate clips, and reranking results.

What is claimed is:

1. A computer-implemented clinical video retrieval system for searching and retrieving clinical videos and clinical text comprising:
a data repository comprising clinical video recordings;
an automatic speech recognition module configured to generate timestamped text transcriptions from the audio content of the clinical video recordings;
an indexing module configured to (i) receive the timestamped text transcriptions, (ii) generate a record comprising a unique identifier, filename, timestamp, transcribed text, and a dense embedding vector of the transcribed text, and (iii) store the record in an index comprising an inverted index and a vector index;
a query processing module configured to receive a text query from a user and generate a dense embedding vector of the text query using a transformer-based embedding model, wherein the query processing module optionally comprises a large language model configured to generate a set of dynamically rephrased query variants of the text query;
a retrieval module configured to perform a keyword-based search using a ranking function on the inverted index and perform a semantic search using k-nearest neighbor (kNN) retrieval on the vector index, wherein results from the keyword-based search and the semantic search are combined using a weighted scoring function, and wherein candidate transcript segments are retrieved for each query variant;
a cross-encoder reranking module configured to apply a cross-encoder reranker to the candidate transcript segments, user query and query variants to compute contextualized similarity scores and sort the candidate transcript segments based on the contextualized similarity scores to produce a ranked list of results;
a video clip generating module configured to extract, for each ranked transcript segment, a video clip from the corresponding clinical video recording, the video clip beginning at the timestamp associated with the transcript segment and having a predefined duration; and
a display module configured to present the ranked list of video clips and associated clinical text segments.

2. The computer-implemented clinical video retrieval system of claim 1, wherein the indexing module is further configured to generate dense embedding vectors using an ensemble of multiple transformer-based embedding models, and to store the multiple embeddings for each transcript segment in the index.

3. The computer-implemented clinical video retrieval system of claim 1, wherein the display module is further configured to present metadata.

4. The computer-implemented clinical video retrieval system of claim 3, wherein the metadata is selected from the group consisting of a filename, a timestamp, and combinations thereof.

5. The computer-implemented clinical video retrieval system of claim 1, wherein the predefined duration starts from the timestamp of the corresponding transcript segment.

6. A computer-aided method for searching and retrieving clinical video clips and clinical text, the method comprising:
(a) receiving a user query comprising a text input;
(b) generating a dense embedding of the user query using at least one transformer-based embedding model;
(c) retrieving, from a repository of clinical video recordings, a plurality of transcript segments, each transcript segment comprising a timestamped transcription of a portion of a clinical video, wherein the retrieval comprises:
(i) performing a keyword-based search using a lexical ranking function to identify transcript segments relevant to the user query;
(ii) performing a semantic search by computing similarity between the dense embedding of the user query and precomputed dense embeddings of the transcript segments to identify semantically relevant transcript segments;
(iii) combining results from the keyword-based search and the semantic search using a weighted scoring function to generate a set of candidate transcript segments;
(d) reranking the set of candidate transcript segments using a cross-encoder model to compute contextualized similarity scores between the user query and each candidate transcript segment, and sorting the candidate transcript segments based on the contextualized similarity scores;
(e) extracting, for each reranked transcript segment, a corresponding clinical video clip from the clinical video recordings, wherein the video clip is generated based on the timestamp associated with the transcript segment;
(f) outputting to a user interface the corresponding clinical video clips as video results, the reranked transcript segments as clinical text results, and combinations thereof.

7. The computer-aided method of claim 6, further comprising enhancing retrieval accuracy by dynamically generating one or more rephrased variants of the user query using a large language model, and repeating steps (b) through (f) for each rephrased variant, wherein results from multiple query variants are combined using a round-robin or interleaving strategy with duplicate removal.

8. The computer-aided method of claim 6, wherein the transcript segments are indexed in a search engine comprising an inverted index for keyword-based search and a vector index for semantic search.

9. The computer-aided method of claim 6, wherein the dense embeddings of the transcript segments are generated using an ensemble of multiple transformer-based embedding models.

10. The computer-aided method of claim 6, wherein the cross-encoder model is selected from the group consisting of MS-MARCO-MiniLM, BAAI General Embeddings (BGE) reranker, nli-deberta-v3-large, stsb-distilroberta-base, jina-reranker-v1-turbo-en, ColBERT, and Qwen2-7B.

11. The computer-aided method of claim 6, wherein the clinical video repository comprises telehealth or telementoring session recordings, and the transcript segments are generated using automatic speech recognition (ASR) software.

12. The computer-aided method of claim 6, wherein the outputted video clips are of a predefined duration.

13. The computer-aided method of claim 12, wherein the predefined duration starts from the timestamp of the corresponding transcript segment.

14. A computer-aided process for indexing clinical video recordings, the process comprising:
receiving a plurality of clinical video recordings, the clinical video recordings comprising audio, video, and presentation materials;
transcribing audio of each clinical video recording of the plurality of clinical video recordings using automatic speech recognition (ASR) software to generate timestamped text transcripts corresponding to segments of the video recordings;
generating dense vector embeddings for each segment of the transcribed text using a transformer-based deep learning model, wherein each dense vector embedding represents semantic content of the corresponding transcript segment;
constructing an index in a search engine system, the index comprising an inverted index for keyword-based search, a vector index, and metadata for each transcript segment including a unique identifier, a filename, a timestamp, transcribed text, and the corresponding dense embedding vector;
storing the index; and
enabling retrieval of relevant video clips.

15. The computer-aided process for indexing clinical video recordings of claim 14, wherein the transformer-based deep learning model is selected from the group consisting of S-PubMedBERT, BGE, E5, BioClinicalBERT, DistilClinicalBERT, TinyClinicalBERT, MedBERT, BlueBERT, Clinical ModernBERT, and combinations thereof.

16. The computer-aided process for indexing clinical video recordings of claim 14, wherein the enabling retrieval of relevant video clips comprises receiving a user text query, generating a dense embedding of the user text query using a transformer-based model, retrieving candidate transcript segments using a combination of keyword-based search and neural vector-based search, reranking the candidate results using a cross-encoder model to compute contextualized similarity scores between the query and each candidate transcript segment, reranking the set of candidate transcript segments using a cross-encoder model to compute contextualized similarity scores between the user query and each candidate transcript segment, and sorting the candidate transcript segments based on the contextualized similarity scores, and outputting a ranked list of video clips, wherein each video clip corresponds to a segment of a clinical video recording starting at the timestamp associated with a top-ranked transcript segment.

17. The computer-aided process for indexing clinical video recordings of claim 14, further comprising dynamically rephrasing the user text query using a large language model to generate a plurality of semantically similar text query variants, performing retrieval and reranking for each semantically similar text query variant.
